(19) United States
(12) Patent Application Publication
(10) Pub. No.: US 2006/0246030 A1
(43) Pub. Date: Nov. 2, 2006

(54) Active ingredient for skin treatments, method for the production thereof and use of the same

(76) Inventor: Jean Paufique, Objat (FR)

Correspondence Address: YOUNG & THOMPSON, 745 SOUTH 23RD STREET, 2ND FLOOR, ARLINGTON, VA, 22202, US

(21) Appl. No.: 10/556,825

(22) PCT Filed: May 12, 2004
(86) PCT No.: PCT/FR04/50189

§ 371 Date: Nov. 15, 2005

(30) Foreign Application Priority Data
May 15, 2003 (FR) 03 05925

(51) Int. Cl. A61K 8/97; A61K 36/49

(52) U.S. Cl. 424/74; 424/771

(57) ABSTRACT

A method for obtaining an active ingredient used in cosmetics includes the following steps: chestnut flour is solubilised in water; enzymatic hydrolysis is carried out; the soluble and insoluble phases are separated by decanting, filtration or centrifuging; and the active phase is concentrated. Also described is the use of the active ingredient, and the active ingredient obtained, for improving the barrier effect on the skin. The active ingredient is characterised by the following parameters: dry matter concentration, pH, total sugar content, and the presence of three glucidic fractions.

This invention relates to the use of a chestnut-based active ingredient, process for obtaining it, and active ingredient obtained for treating dry skin by means of multiple actions generated by said active ingredient.

The invention also covers the active ingredient that is obtained as well as the compositions that integrate it.

The dry skin phenomenon has been known for a long time and in particular it is extremely visible and noted by the individuals that experience it.

This problem of dry skin is often associated with a rough, bumpy skin with a scaly appearance as opposed to hydrated skin whose appearance is smooth and soft.

One simple and commonly used solution consists in ensuring a cutaneous hydration, but such a solution is not satisfactory.

On the surface, the skin comprises the outermost layer that is named the stratum corneum.

This layer is particularly important because it protects against physical and chemical attacks, and it plays a barrier role for regulating the water loss and the penetration of xenobiotics. In addition, this layer ensures a mechanical protection.

This layer consists of:
corneocytes, and
intercellular lipids.

During aging and during dermatological afflictions, the barrier function of the skin may be affected.

During a state of dryness of the skin, the following different factors are involved:
Deregulation of exfoliation,
Inability to hold water, due in particular to a change in the processes of synthesis and degradation of the profilaggrin, and
Deficiency of lipids that play an essential role in maintaining an epidermal barrier, in particular the ceramides.

The process according to this invention makes it possible to obtain an active ingredient starting from the chestnut that acts on the different ingredients involved in maintaining homeostasis of the horny layer.

This same active ingredient normalizes the cohesion/exfoliation balance and the epidermal differentiation and restores the synthesis mechanisms of the epidermal lipids.

The process is now described by way of its different stages that make it possible to obtain an active ingredient with multiple effects.

The description is completed by different in-vitro and in-vivo tests that make it possible to highlight these effects.

### 1/PROCESS FOR OBTAINING THE ACTIVE INGREDIENTS

Solubilization of chestnut meal in water at a rate of at least 100 g/l,

Enzymatic hydrolysis with one or more carbohydrases, in a successive or simultaneous manner,

Separation of soluble and insoluble phases by decanting, filtration or centrifuging, and

Concentration of the soluble active phase.

The carbohydrase is preferably used at a rate of at least 0.1%.

As for the term “meal” used in this description, it is in no way limiting relative to a grain size that is given or considered common. This term of meal is commonly used to distinguish a powder, in this case a chestnut powder.

### 2/CHARACTERIZATION OF THE ACTIVE INGREDIENT ACCORDING TO THE INVENTION

2-1/Level of Dry Material

The level of dry material is obtained by passage in an oven at 105° C. of a 10 g sample of product until a constant weight is obtained.

The level of dry material is between 10 and 300 g/l, more particularly between 80 and 120 g/l.

2-2/Measurement of the pH

The pH, determined by potentiometric measurement, leads to values of between 4.0 and 8.0, specifically between 5.0 and 6.0.

2-3/Determination of the Total Sugar Content

The dosage is carried out by the Dubois method (Dubois, M. et al., (1956), Analytical Chemistry, 28, No. 3, pp. 350-356).

The measurement is carried out by measuring the optical density of the coloration taken by the reducing sugars in the presence of concentrated sulfuric acid.

This optical density is related to a standard range of mannose-glucose-galactose.

The results that are obtained provide sugar levels of between 9 and 275 g/l and more particularly between 72 and 100 g/l.

2-4/Characterization of the Glucide Fraction:

The process that is used is the thin layer chromatography of the glucide fraction of the active ingredient of this invention.

The conditions of chromatography are:
Acetic acid, butanol, water in a 1/2/1 ratio as eluant
Double migration
20% H₂SO₄ and 0.1% orcinol as a disclosure mixture

The analysis of the chromatography indicates the presence of three fractions:
A polysaccharide fraction: rhamnogalacturonan,
An oligosaccharide fraction with a high degree of polymerization and free uronic acids, and
A mono- and oligosaccharide fraction of a low degree of polymerization.

### 3/EFFECTS OF THE ACTIVE INGREDIENT ON THE BARRIER FUNCTION

3-1/Effect of the Active Ingredient on the Differentiation of Human Keratinocytes:

The cells migrate toward the surface and the keratinocytes are gradually transformed into keratinized cells named corneocytes that are eliminated by exfoliation.

To maintain a constant thickness, the renewal is carried out by cellular division from stock cells.

3-1-1/Study of the Synthesis of Involucrin

The formation of the horny jacket begins starting from precursor proteins, in particular involucrin. A membrane enzyme, the transglutaminase, forms covalent bonds between the proteins.

The involucrin is a protein that constitutes the protein skeleton of the plasmic membrane of the corneocytes.

To determine the action of the active ingredient according to the invention, the effect of this active ingredient on the expression of RNA messengers that code for involucrin is analyzed. For this purpose, human keratinocytes are incubated in the presence of the active ingredient dosed at 0.5%, 1% and 2%. The total RNA are extracted, and the percentage of mRNA that codes for the involucrin relative to a control is determined.

The results that are obtained are summed up in the table below.

| | % of Messenger RNA That Codes for |
|---|---|
| | Involucrin/Control |
| Control | 100 |
| Active Ingredient According to the | 111 |
| Invention Dosed at 0.5% | |
| Active Ingredient According to the | 117 |
| Invention Dosed at 1.0% | |
| Active Ingredient According to the | 121 |
| Invention Dosed at 2.0% | |

It is noted that at 2%, the active ingredient according to the invention makes it possible to increase the expression of the mRNA that codes for the synthesis of involucrin by 21%.

3-1-2/Study of the Synthesis of the Profilaggrin

The horny jacket imparts to corneocytes the stiffness and therefore the mechanical resistance of the stratum corneum. The fibrous material that is substituted with cytoplasm and the keratinocyte nucleus is formed from profilaggrin. This profilaggrin is transformed into filaggrin that makes possible the aggregation of the cytokeratin filaments. The degradation releases hygroscopic substances that have a significant power for securing the water.

It is therefore necessary to determine as above the ratio of the expression of RNA messengers coding for the profilaggrin relative to a control.

This analysis is carried out from human keratinocytes that are incubated in the presence of 0.5%, 1% and 2% of active ingredient.

| | % of Messenger RNA That Codes for |
|---|---|
| | Profilaggrin/Control |
| Control | 100 |
| Active Ingredient According to the | 116 ± 8 |
| Invention Dosed at 0.5% | |
| Active Ingredient According to the | 137 ± 9 |
| Invention Dosed at 1.0% | |
| Active Ingredient According to the | 147 ± 12 |
| Invention Dosed at 2.0% | |

It is noted that from 2%, the active ingredient according to the invention also makes it possible to increase the expression of the mRNA that codes for the synthesis of profilaggrin in proportions of 47%.

It is therefore possible to conclude that the active ingredient promotes cellular differentiation.

3-1-3/Study of the Synthesis of Cadherin-E

The cadherins play a role in attachment between the cells, but also in morphogenesis and the monitoring of the cellular differentiation.

Cadherin-E is located in cellular layers of the epidermis and in particular in differentiated layers.

The active ingredient according to the invention has an effect on the synthesis of cadherin-E, which the following test, which consists in treating keratinocytes with the active ingredient according to the invention at 0.5%, 1% and 2%, demonstrates.

The total proteins are dosed, and the evolution of the cadherin-E level relative to the control is determined.

| | Level of |
|---|---|
| | Cadherin-E/Control |
| Control | 100 |
| Active Ingredient According to the | 111 ± 5 |
| Invention Dosed at 0.5% | |
| Active Ingredient According to the | 127 ± 8 |
| Invention Dosed at 1.0% | |
| Active Ingredient According to the | 136 ± 10 |
| Invention Dosed at 2.0% | |

The active ingredient promotes the synthesis of cadherin-E by 36% for a 2% dosage.

3-2/Effect of the Active Ingredient on the Synthesis of the Epidermal Lipids:

The lipids play an essential role in the barrier function.

It is therefore necessary to analyze the actions of the active ingredient according to the invention on these lipids.

3-2-1/Study of the Ceramide Content

The ceramides [which] are one of the components of the intercellular cement of the horny layers. The ceramides are obtained from phospholipids and glyceryl-ceramides that are dephosphorylated or hydrolyzed.

Skin explants are treated to extract lipids, and these samples are analyzed, whereby the ceramides are separated by thin layer chromatography.

The following results show that the active ingredient according to this invention acts on the synthesis of the ceramides since it increases it from 40%.

| | Content of |
|---|---|
| | Ceramides/Placebo |
| Placebo | |
| Active Ingredient According to the | +15 ± 5 |
| Invention at 3.0% | |
| Active Ingredient According to the | +40 ± 13 |
| Invention at 5.0% | |

3-2-2/Study of the Expression of the Synthesis Enzymes of the Lipids

The mRNA levels of the enzymes such as FAS (fatty acid synthase) and STP (serine palmitoyl transferase) increase during the barrier repair process.

Human keratinocytes are incubated in the presence of 0.5%, 1% and 2%.

The cells are recovered, and the total RNA are extracted.

The expression percentage of the mRNA of FAS and STP relative to a control is determined.

A significant increase of expression of enzymes for synthesis of lipids is noted in the following tables.

| | Percentage of |
|---|---|
| | mRNA That Codes for |
| | the FAS/Control |
| Control | 100 |
| Active Ingredient According to the | 101 ± 6 |
| Invention at 0.5% | |
| Active Ingredient According to the | 129 ± 6 |
| Invention at 1.0% | |
| Active Ingredient According to the | 146 ± 6 |
| Invention at 2.0% | |

| | Percentage of |
|---|---|
| | mRNA That Codes |
| | for the STP/Control |
| Control | 100 |
| Active Ingredient According to the | 111 ± 5 |
| Invention at 0.5% | |
| Active Ingredient According to the | 125 ± 9 |
| Invention at 1.0% | |
| Active Ingredient According to the | 132 ± 8 |
| Invention at 2.0% | |

3-2-3/Study on Volunteers with Regard to the Synthesis of Epidermal Lipids

Tender zones are determined in volunteers, and they are treated: one with a placebo and the other with the active ingredient according to the invention, formulated at 4% in emulsion.

After a twice-daily treatment for 7 days, the epidermal lipids are sampled with an alcoholic solution.

The effects of the active ingredient on the synthesis of lipids, more particularly the ceramides, are determined.

| | |
|---|---|
| Active | |
| Ingredient | |
| at 4% | Placebo |

| | JO | J7 | JO | J7 |
|---|---|---|---|---|
| Average | 7.58 | 10.63 | 7.63 | 10.06 |

At 4%, the active ingredient increases the level of caramides of the stratum corneum by 8%. This promotes the restoration of the lipid barrier of the stratum corneum.

3-3/Effect of the Active Ingredient on the Synthesis of Desmoglein-1:

The desmogleins and the desmocollins are glycoproteins of the desmosomal cadherin family that take part in the formation of interkeratinocyte junctions.

Desmoglein-1 is one of the three isoforms and is located only in the surface layers of the epidermis.

The more the synthesis of this glycoprotein decreases, the more the exfoliation is promoted, which prevents the phenomenon of dry skin.

The following test makes it possible to determine the effect of the active ingredient on the synthesis of this isoform.

A treatment of human keratinocytes with 0.5%, 1% and 2% is carried out.

The dosage of the total proteins is carried out, and the specific amount of this glycoprotein is determined.

| | | Variation of |
|---|---|---|
| | Level of | the Level of |
| | Desmoglein- | Desmoglein-1/ |
| | 1/Control | Control |
| Control | 100 | — |
| Active Ingredient at 0.5% | 94 | −6 ± 4 |
| Active Ingredient at 1.0% | 86 | −14 ± 6 |
| Active Ingredient at 2.0% | 79 | −21 ± 6 |

By reducing by 21% the synthesis of the desmoglein-1 with a 2% dosage, the active ingredient promotes the exfoliation process.

3-4/Effect of the Active Ingredient on the Insignificant Loss of Water:

The exfoliation process is essential in the preservation of the stratum corneum by promoting the hydration of the skin.

To measure the effect of the active ingredient on the reinforcement of the barrier effect, the pressure gradient of the water vapor layer that surrounds the skin is measured.

The application of lauryl sodium sulfate on the skin so as to promote water losses, and the water losses in zones attacked by lauryl sodium sulfate and a zone that is untreated or attacked and treated with the placebo emulsion are compared.

| | Δ (%) | ΔΔ (%) |
|---|---|---|
| Untreated Control Zone | 103 | |
| Placebo | 102 | |
| Zone Treated with the | 85 | −17 |
| Active Ingredient | | |
| According to the Invention | | |
| at 4% | | |

The negligible water loss is decreased to 17% using the active ingredient dosed at 4%.

3-5/Effect of the Active Ingredient on the Effectiveness of the SCCE:

The purpose is to determine the corneocytic turn-over. The activity of the SCCE (stratum corneum chymotrpysin enzyme) is measured.

A strong mechanical attack on the stratum corneum is carried out by stripping.

Three attacked zones, one not treated, the other treated with a placebo, and a last zone treated with the active ingredient at 4%, are determined.

This SCCE enzyme is recovered in each of the zones, and it is dosed by spectrophotometric dosage.

| | % SCCE | % |
|---|---|---|
| | Variation/Untreated | Variation/ |
| | Attacked Zone | Placebo |
| Untreated Attacked Zone | — | — |
| Placebo-Treated Attacked | −42% | — |
| Zone | | |
| Attacked Zone Treated with | +40% | 82% |
| the Active Ingredient Dosed | | |
| at 4% | | |

It is noted that after a strong mechanical attack, the activity of the SCCE is significantly increased.

The negligible water loss is also measured in these same zones that are: one not treated, the other treated with a placebo, and a last zone treated with the active ingredient at 4%.

| | % of Recovery of the | % of |
|---|---|---|
| | Negligible Water Loss | Recovery/Placebo |
| Untreated Attacked Zone | +84% | — |
| Placebo Treated Attacked | +84% | — |
| Zone | | |
| Attacked Zone Treated with | +90% | +6% |
| the Active Ingredient Dosed | | |
| at 4% | | |

The negligible water loss is decreased in a statistically significant way, the Student's test on paired data being significant.

Thus, the active ingredient according to this invention intensifies the natural repair cycle of the stratum corneum by increasing the effectiveness of the SCCE and by maximizing the repair potential of the barrier function.

The active ingredient is used with any adapted cosmetic galenical form such as an aqueous or alcoholic emulsion, a lotion, a cream with an aqueous or fatty base, or an ointment, at a rate of 0.1 to 20%.

1. A method of improving a barrier effect on skin comprising applying an effective amount of an active ingredient that is obtained from chestnut meal, associated with any adapted cosmetic galenical form.

2. The method according to claim 1, wherein the application makes possible an improvement of the synthesis of involucrin on the skin.

3. The method according to claim 1, wherein the application makes possible an improvement of the synthesis of profilaggrin on the skin.

4. The method according to claim 1, wherein the application promotes synthesis of cadherin-E on the skin.

5. The method according to claim 1, wherein the application increases synthesis of the ceramides on a horny layer.

6. The method according to claim 1, wherein the application makes it possible to increase expression of enzymes for synthesis of lipids.

7. The method according to claim 1, wherein the application makes possible a reduction in activity of the glycoproteins of the family of desmosomal cadherins so as to promote exfoliation.

8. The method according to claim 1, wherein the application makes possible a reduction of negligible water loss.

9. A process for obtaining the active ingredient that is used according to claim 1, comprising the steps of:
Solubilizing chestnut meal in water,
Performing enzymatic hydrolysis,
Separating soluble and insoluble phases by at least one of decanting, filtering and centrifuging, and
Concentrating the active phase.

10. Production process according to claim 9, wherein the solubilization is carried out with at least 100 g/l of chestnut meal.

11. Production process according to claim 9, wherein the enzymatic hydrolysis is carried out in the presence of at least one carbohydrase.

12. Active ingredient obtained according to the process of claim 9, characterized by the following parameters:
Level of dry material of between 10 and 300 g/l,
pH of between 4.0 and 8.0
Total sugar content of between 9 and 275 g/l, and
Presence of three glucide fractions:
Polysaccharide fraction: rhamnogalacturonan
Oligosaccharide fraction with a high degree of polymerization and free uronic acids, and
A mono- and oligosaccharide fraction of a low degree of polymerization.

13. Active ingredient that is obtained according to the process of claim 9, characterized by the following parameters:
Level of dry material of between 80 and 120 g/l,
pH of between 5.0 and 6.0,
Total sugar content of between 72 and 110 g/l.

14. The method of claim 1, wherein the cosmetic galenical form is one selected from the group consisting of an aqueous emulsion, an alcoholic emulsion, a lotion, a cream with an aqueous base, a cream with a fatty base, and an ointment

15. The method according to claim 6, wherein the lipids consist of at least one selected from the group consisting of the fatty acid synthase, serine palmitoyl transferase, and the synthesis of epidermal lipids on the skin.

16. The method according to claim 8, wherein the application reduces the negligible water loss by increasing the activity of the chymotrysin stratum corneum enzyme.

17. Production process according to claim 11, wherein the enzymatic hydrolysis is carried out in the presence of the at least one carbohydrase at a rate of at least 0.1%.
